(19) United States
(12) Patent Application Publication
(10) Pub. No.: US 2021/0379245 A1
(43) Pub. Date: Dec. 9, 2021

(54) Soluble Extracellular Matrix Composition and Method for Intravascular Delivery

(71) Applicant: The Regents of the University of California, Oakland, CA (US)

(72) Inventors: Karen Christman, San Diego, CA (US); Martin Spang, San Diego, CA (US); Gregory Grover, La Jolla, CA (US)

(21) Appl. No.: 17/286,534

(22) PCT Filed: Oct. 25, 2019
(86) PCT No.: PCT/US2019/058052

§ 371 Date: Apr. 19, 2021

Related U.S. Application Data
(60) Provisional application No. 62/750,303, filed on Oct. 25, 2018.

(51) Int. Cl. A61L 27/36; A61L 27/54; A61K 38/01

(52) U.S. Cl. CPC A61L 27/3633; A61L 27/3683; A61L 2400/06; A61K 38/014; A61L 27/54

(57) ABSTRACT

Compositions and methods for their manufacture and use are provided comprising a soluble extracellular matrix fraction for intravascular delivery which forms a gel or coating in situ for treatment of myocardial infarction and ischemia in a variety of tissues and endothelial injury/dysfunction.

### CROSS REFERENCE TO RELATED APPLICATIONS

This application claims the priority benefit of U.S. Provisional Application No. 62/750,303, filed Oct. 25, 2018, which application is incorporated herein by reference.

### GOVERNMENT SPONSORSHIP

This invention was made with government support under grant No. HL113468 awarded by the National Institutes of Health (NIH). The government has certain rights in the invention.

### TECHNICAL FIELD

The present invention relates to an infusable soluble extracellular matrix composition and therapy for minimally invasive delivery to tissues/organs/cells, including ischemic or injured heart, brain, skeletal muscle, blood vessels, and endothelial cells.

### BACKGROUND

Extracellular matrix therapies include native tissue components providing a scaffold for tissue regeneration. Current decellularized extracellular matrix therapies are restricted to patches or direct injections. No extracellular matrix therapy is capable of intravascular/infusion delivery to a tissue of interest. ECM hydrogels made from decellularized and digested tissue have been created, but they are not fully soluble or transparent colloids. They are translucent suspensions that contain both a soluble and non-soluble component (Freytes et al, 2008, Singelyn et al, 2009)) which prevents the material from passing from the blood stream through leaky vasculature (which occurs in ischemic tissue such as acute myocardial infarction, stroke, cancers, etc (Nguyen et al, 2015, Dvorak et al, 1988, Yuan et al, 1995)) into a tissue, lining the leaky vasculature, or filling the pores of leaky vasculature.

Myocardial infarction (MI) is characterized by ischemic necrosis of the myocardium that progresses over time, leading to negative left ventricular (LV) remodeling and eventual heart failure. The current standard of care does not address this ischemic damage. A minimally-invasive tissue engineering therapy could repair the heart post-MI. A number of stem cell and growth factor therapies have reached clinical trials; however, these therapies have displayed poor efficacy, likely due to the inadequate retention of non-encapsulated therapies.

Extracellular matrix (ECM) hydrogels have shown great promise in the field of cardiac tissue engineering; in particular, a tissue-specific hydrogel derived from decellularized porcine myocardium, termed myocardial matrix (MM), has shown increases in cardiac muscle, neovascularization of the infarct region, and improved regional and global cardiac function in MI models [2-4]. Furthermore, mechanisms of repair were investigated through whole transcriptome analysis on RNA isolated from the infarct region of rat hearts injected with MM, indicating upregulated pathways associated with cardiac repair (e.g. neovascularization and heart development) and downregulated pathways associated with negative LV remodeling (e.g. hypertrophy, apoptosis, and fibrosis) [6]. This material was evaluated in a Phase I trial in post-MI patients using transendocardial injections 60 days to 3 years post-MI (ClinicalTrials.gov Identifier: NCT02305602). However, cardiomyocyte death and negative LV remodeling are processes that commence within minutes to days after the MI [26, 27]. Current delivery of the MM is limited to transendocardial catheter injections, as it not amenable to intracoronary infusion because it contains submicron particles that are too large to pass through the leaky coronary vasculature into the infarct. Furthermore, a transendocardial injection is a specialized medical technique with some safety concerns of ventricular rupture and arrhythmias within the first week post-MI [7, 8]. This prevents delivery of the MM within the critical therapeutic window following an acute MI.

Intracoronary infusion is an alternative approach to transendocardial injections. Intracoronary delivery is feasible in an acute MI, as it can accompany a balloon angioplasty that typically occurs shortly after the patient is admitted to a hospital. Such a technique is standard in interventional cardiology and does not require specialized training. Intracoronary infusion takes advantage of the leaky vasculature following an acute MI permitting a biomaterial to pass through the coronary vasculature and enter the infarct region [5]. The intracoronary delivery of a biomaterial has shown feasibility with alginate hydrogels in a porcine MI model [9] and has progressed to Phase II clinical trials (ClinicalTrials.gov Identifier: NCT01226563). However, this material did not show significant improvements in cardiac function, possibly due to the limited bioactivity of alginate [10].

### SUMMARY OF THE INVENTION

In embodiments, the invention provides a method of preparing a soluble extracellular matrix (ECM) composition, comprising enzymatically digesting ECM material with an acid protease, such as pepsin; neutralizing the digested ECM material in liquid to a pH of 7.0-8.0; processing the liquid ECM to produce soluble and insoluble fractions; and separating at least a portion of the soluble fraction from the insoluble fraction, to yield a soluble ECM composition.

In embodiments, the invention provides that processing the liquid ECM to produce soluble and insoluble fractions is achieved by centrifugation. In embodiments, the invention provides that the soluble ECM composition is dialyzed and/or filtered to remove insoluble materials. In embodiments, the invention provides that the soluble ECM composition is further lyophilized for storage and re-hydrated for use.

In embodiments, the soluble ECM composition is substantially isolated from ECM solids in the liquid ECM. In embodiments, the soluble ECM composition is more transparent than the digested unseparated ECM material. In embodiments, the soluble ECM composition includes transparent ECM colloids, which can pass through a 0.25 μm filter.

In embodiments, the invention provides a method of treating a subject in need thereof comprising administering to the subject an effective amount of the soluble ECM composition, to promote tissue repair or cell recruitment. In embodiments, the infusion is through a catheter, intravenously, or intravascularly. In embodiments, the invention provides that when delivered in vivo, the soluble fraction will then form a gel in tissue. In embodiments, the soluble fraction will coat the lining of blood vessels. In embodiments, the soluble fraction will fill the pores, fenestrations, endothelial disruptions, open intercellular junctions, or gaps of leaky or damaged vasculature.

In embodiments, the invention provides that the soluble fraction of ECM is further crosslinked with glutaraldehye, formaldehyde, bis-NHS molecules, or other crosslinkers. In embodiments, the invention provides that the soluble fraction of ECM is combined and/or crosslinked with a synthetic polymer or biologically derived material. In embodiments, the invention provides that the soluble fraction of ECM is combined with cells, peptides, proteins, DNA, drugs, nanoparticles, antibiotics, growth factors, nutrients, survival promoting additives, proteoglycans, and/or glycosaminoglycans.

In embodiments, the invention provides that the soluble fraction of ECM is used in combination with above described components for endogenous cell ingrowth, angiogenesis, and regeneration. In embodiments, the invention provides that the soluble fraction of ECM is used in combination with above described components as a matrix to change mechanical properties of the tissue. In embodiments, the invention provides that the soluble fraction of ECM is delivered with cells alone or in combination with above described components for regenerating or repairing damaged tissue.

In embodiments, the invention provides that after adjusting the concentration and/or sterile filtration, the soluble fraction of ECM can be lyophilized and stored frozen (e.g. −20 C, −80 C) for at least 3 months. The soluble ECM composition, or fraction, can then be resuspended and/or sterile filtrated prior to injection or infusion.

In embodiments, the invention provides that after adjusting the concentration and/or sterile filtration, the soluble fraction of ECM can be lyophilized and stored in the refrigerator (e.g. 4 C) for at least 3 months. The soluble ECM fraction can then be resuspended and/or sterile filtrated prior to injection or infusion.

In embodiments, the invention provides that after adjusting the concentration and/or sterile filtration, the soluble fraction of ECM can be lyophilized and stored at room temperature for at least 3 months. The soluble ECM fraction can then be resuspended and/or sterile filtrated prior to injection or infusion.

In embodiments, the invention provides that the method of separating at least a portion of the soluble and insoluble fractions of liquid extracellular matrix (pre-gel solution) can be performed by high-speed centrifugation, dialysis, filtration, or adjusting pH or salinity. In embodiments, the separation of soluble fraction is performed by removing at least a portion of solids from the ECM material. In embodiments, the separation of soluble fractions is performed with a filter having a size limitation of less than 1 μm, 0.5 μm, 0.25 μm, 0.22 μm, or 0.2 μm. In embodiments, the invention provides a soluble ECM composition that is derived from decellularized tissue and processed to isolate a soluble fraction prior to gelation in vivo. In embodiments, the invention provides that the composition of soluble ECM is prepared for intravascular infusion.

In embodiments, the invention provides that the composition of soluble extracellular matrix is derived from human, animal, embryonic, and/or fetal tissue sources. In embodiments, the invention provides that the composition of soluble extracellular matrix is derived from heart, brain, bladder, small intestine, skeletal muscle, kidney, liver, lung, blood vessels, and other tissues/organs tissue sources.

In embodiments, the invention provides a method for treating acute myocardial infarction comprising injecting or infusing in a subject in need with myocardial infarction an effective amount of a composition comprising soluble decellularized extracellular matrix derived from muscle tissue.

In embodiments, the invention provides that said soluble ECM composition is delivered intravascularly by infusion. In embodiments, the invention provides that said soluble ECM composition is delivered by intracoronary infusion with a balloon infusion catheter. In embodiments, the invention provides that said soluble ECM composition transitions to a gel form in tissue after delivery. In embodiments, the invention provides that said soluble ECM composition transitions to form a coating on the endothelium of injured blood vessels after delivery. In embodiments, the invention provides that said soluble ECM composition degrades within one to 14 days following injection or infusion.

In embodiments, the invention provides that the injection or infusion of said composition repairs damage to cardiac muscle sustained by said subject, such as a myocardial infarction. In embodiments, the invention provides that the injection or infusion of said composition is used to treat muscular or neurological damage caused by disease, trauma, stroke and/or ischemia in said subject. In embodiments, the invention provides that said effective amount is an amount that increases blood flow, increases viable tissue mass, or induces new vascular formation in the area of the injection or infusion of the subject. In embodiments, the invention provides that said effective amount is an amount that promotes cell survival, reduces inflammation, and repairs damaged vasculature in the area of the injection or infusion of the subject.

### BRIEF DESCRIPTION OF THE DRAWINGS

FIG. 1A-1F show generation of soluble myocardial matrix. FIG. 1A shows an isolated left ventricular myocardium is cut into pieces. FIG. 1B shows decellularized after continuous agitation in 1% sodium dodecyl sulfate. FIG. 1C shows lyophilized and milled into fine powder. FIG. 1D shows partially digested myocardial matrix. (E) Fractionated myocardial matrix after centrifugation, (1) SolMM fraction in the supernatant and (2) insoluble pellet. (F) SolMM hydrogel post-subcutaneous injection. Images (A-D) were taken from [3].

FIG. 2 shows PAGE comparing protein distribution of ladder (Full-Range RPN800E, lane 1), collagen (lane 2), myocardial matrix (lane 3), and soluble myocardial matrix (lane 4).

FIG. 3 shows distribution and retention of SolMM (red greyscales) at 12 hours post-intracoronary injection of 200 μL of 10 mg/mL SolMM in an ischemia-reperfusion rat model. (Left) Short axis view of infarcted heart stained with hematoxylin & eosin, infarct spanning across lower half of the heart, scale bar 3 mm; (Right) Inset on infarcted myocardium displaying SolMM micro-scale gels throughout the infarcted myocardium, scale bar 200 μm.

FIG. 4 shows distribution and retention of SolMM (red greyscales) 1 hour following intracoronary infusion in a porcine ischemia-reperfusion model. (Left) Short axis gross histology of infarcted pig heart. Infarct outlined in blue greyscales. (Right) Infarcted myocardium displaying SolMM micro-gels throughout the infarcted myocardium.

FIG. 5 shows mitigated negative left ventricular remodeling (preserved EDV and ESV) following intracoronary infusions of SolMM in an ischemia-reperfusion model 24 hours and 5 weeks post-infusion. EDV end diastolic volume, ESV end systolic volume, EF—ejection fraction, SolMM (blue greyscales squares), saline (red greyscales circles). N=10-11 per group.

FIG. 6 shows increased infarct arteriole density in SolMM infused rats 5 weeks post-infusion and ischemia-reperfusion. Arterioles were identified by co-staining for alpha-smooth muscle actin and isolection and manually traced in ImageJ. N=10-11 per group.

FIG. 7 shows decreased cardiomyocyte apoptosis the infarct border zone in SolMM infused rats 3 days post-infusion and ischemia-reperfusion. Tissue was stained with alpha-actinin for cardiomyocytes and cleaved-caspase 3 for apoptosis. Apoptotic cardiomyocytes were manually counted in ImageJ. N=5-6 per group.

FIG. 8 shows relative gene expression changes in SolMM infused rats 1 day post-infusion and ischemia-reperfusion. Gene expression was measured by RT-qPCR using RNA isolated from the LV free wall. Day 1 gene expression suggests increased angiogenic and reactive oxygen species metabolic pathways. N=5-6 per group.

FIG. 9 shows relative gene expression changes in SolMM infused rats 3 days post-infusion and ischemia-reperfusion. Gene expression was measured by RT-qPCR using RNA isolated from the LV free wall. Day 3 gene expression suggests decreased apoptosis/necrosis and fibrosis pathways. LRG1 has been suggested in angiogenic pathways, and LRG1 downregulation is implicated in fibrosis. N=5-6 per group.

FIG. 10 shows confocal imaging of soluble matrix (red greyscales) and isolectin for endothelial cells (green greyscales) following an infusion of soluble matrix in an ischemia-reperfusion rat model. The panels are sequential images from a z-stack. Soluble matrix is coating the inside of a small (approx 5 μm diameter) capillary, but the soluble matrix is not completely blocking the lumen.

FIG. 11 shows confocal imaging of soluble matrix (red greyscales) and isolectin for endothelial cells (green greyscales) following an infusion of soluble matrix in an ischemia-reperfusion rat model. Soluble matrix overlaps endothelial cells and does not block the lumen of the vessel.

FIG. 12 show soluble matrix retention in soluble matrix infused hearts 24 hours post-infusion and ischemia-reperfusion. From left to right, hearts were infused with 1) saline, 2) 10 mg/ml soluble matrix conjugated with Vivo Tag 750, 3) 10 mg/ml trilysine conjugated with Vivo Tag 750, 4) 10 mg/ml soluble matrix conjugated with Vivo Tag 750. Trilysine was used as a small peptide control and showed minimal heart retention.

FIG. 13 shows a scanning electron microscope image of soluble matrix hydrogel. Scale bar left image 20 μm, scale bar right image 5 μm.

FIG. 14 shows dynamic light scattering data for soluble matrix (SolMM) and full matrix (MM) at 1:50 dilutions (1.0 mg/ml & 0.6 mg/ml respectively), showing soluble matrix particles less than 100 nm in diameter whereas full matrix has larger particles.

FIG. 15 shows dynamic light scattering data for soluble matrix (SolMM) at 1:10 and 1:100 dilutions (1.0 mg/ml & 0.1 mg/ml respectively), showing particles less than 100 nm in diameter.

FIG. 16 shows absorbance (left) and transmittance (right) of saline, soluble matrix (SolMM), and full matrix (MM).

FIG. 17 shows relative absorbance (left) and transmittance (right) of soluble matrix (SolMM) and full matrix (MM).

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

In embodiments, the invention provides a method of preparing one or more biologically active soluble fractions of extracellular matrix (ECM) for therapeutic delivery, comprising:
a. partially or completely digesting with an acid protease, such as pepsin, decellularized ECM prepared from a tissue;
b. neutralizing the digested ECM material to a pH of 7.0-8.0;
c. processing the liquid ECM (pre-gel solution) to produce soluble and insoluble fractions; and
d. separating at least a portion of the insoluble fraction away from the soluble fraction, to yield a soluble ECM composition.

In embodiments, the invention provides that the soluble ECM composition is further lyophilized, dialyzed, and/or filtered. In embodiments, the invention provides that the soluble ECM composition is re-hydrated following lyophilization.

In embodiments, the invention provides a method of treating a subject in need thereof comprising administering to the subject an effective amount of an intravascular infusion of a soluble ECM composition, to promote organ, tissue, or cell repair or cell recruitment. In embodiments, the infusion is through a catheter, intravenously, or intravascularly. In embodiments, the invention provides that when delivered in vivo, the soluble ECM composition will then form a gel in and/or around the microvasculature of the tissue.

In embodiments, the invention provides a method for treating acute myocardial infarction comprising injecting or infusing in a subject in need with myocardial infarction an effective amount of a composition comprising soluble decellularized extracellular matrix derived from muscle tissue.

In embodiments, the invention provides that said composition is delivered intravascularly. In embodiments, the invention provides that said composition is delivered with a balloon infusion catheter. In embodiments, the invention provides that said composition transitions to a gel form in tissue after delivery. In embodiments, the invention provides that said composition degrades within one to 14 days following injection or infusion. In embodiments, the invention provides that the injection or infusion of said composition repairs damage to cardiac muscle sustained by said subject. In embodiments, the invention provides that the injection or infusion of said composition repairs damage in non-cardiac tissues caused by trauma or ischemia in said subject.

In embodiments, the invention provides that the effective amount is an amount that increases blood flow, increases viable tissue mass, or induces new vascular formation in the area of the injection or infusion of the subject.

For human therapy, there are many source species for the extracellular matrix: e.g., human, porcine, bovine, goat, mouse, rat, rabbit, chicken, and other animal sources. Furthermore, there are many tissue sources: e.g., heart, brain, bladder, small intestine, skeletal muscle, kidney, liver, lung, blood vessels and other tissues and organs.

In embodiments, the tissue is first decellularized, leaving only the extracellular matrix such as disclosed in U.S. Patent Publication US2013/0251687, for example, which is incorporated by reference in its entirety. The matrix is then lyophilized, ground or pulverized into a fine powder, solubilized with pepsin or other enzymes, and subsequently neutralized and buffered as previously reported. Following neutralization, the digestion (pre-gel solution) is fractionated to separate soluble and insoluble fractions. Processing the separation of soluble and insoluble fractions may be achieved by centrifugation, dialysis, filtration, or adjusting pH or salinity. The soluble fraction can be dialyzed to remove salts, lyophilized, and resuspended to adjust ECM concentration. ECM can be sterile filtered, lyophilized, and stored in sterile containers. ECM can be resuspended to appropriate/physiological concentration for infusion.

A soluble ECM composition refers to extracellular matrix material which has been decellularized, lyophilized, ground, and digested and having at least a portion of the solid components removed therefrom. In embodiments, a soluble ECM composition is obtained from centrifugation supernatant. In embodiments, soluble ECM composition is able to pass through a filter size of less than 1 μm, 500 nm, 250 nm, 220 nm, or 200 nm. The soluble ECM composition having at least a portion of solid ECM components with which it naturally occurs removed therefrom is a more transparent material than before removal of the ECM solids. However, it is to be understood that some degree of insoluble small particulate matter, such as ECM colloids, may still be present in the soluble ECM composition. A soluble ECM composition has been substantially isolated when at least 50%, 60%, 70%, 80%, 90%, 95%, 98% or 99% of the naturally occurring ECM solids by volume have been removed therefrom.

After adjusting concentration and/or sterile filtration, the soluble ECM composition can be lyophilized and stored frozen (e.g. −20 C, −80 C) for at least 3 months. The soluble ECM composition can then be rehydrated with sterile water prior to injection or infusion.

The soluble ECM composition can be infused through a catheter, delivered intravenously, or by intravascular infusion with or without a balloon. The soluble ECM composition can pass through damaged leaky vasculature, such as that found in an acute myocardial infarction, stroke, other ischemic tissues, tumors, etc. Once in the tissue, the soluble ECM composition will then form into a gel.

The soluble ECM composition can be infused through a catheter, delivered intravenously, or by intravascular infusion with or without a balloon. The soluble ECM composition can assemble into a coating on the lining of or fill the pores of leaky vasculature, such as that found in an acute myocardial infarction, stroke, other ischemic tissues, tumors, tissues suffering trauma, etc.

The soluble ECM composition gel can be crosslinked with glutaraldehye, formaldehyde, bis-NHS molecules, or other crosslinkers. The soluble ECM composition can be combined with cells, peptides, proteins, DNA, drugs, nutrients, survival promoting additives, proteoglycans, and/or glycosaminolycans. The soluble ECM composition can be combined and/or crosslinked with a synthetic polymer. The soluble ECM composition can be used alone or in combination with above described components for endogenous cell ingrowth, angiogenesis, and regeneration. The soluble ECM composition can be use alone or in combination with above described components as a matrix to change mechanical properties of the tissue. The soluble ECM composition can be delivered with cells alone or in combination with above described components for regenerating damaged tissue.

The soluble ECM composition can be used for tissue repair following tissue injury such as due to myocardial infarction, stroke, traumatic brain injury, peripheral artery disease, liver chirrosis, cancerous tumors, or renal injury. The soluble ECM composition can be used alone or act as a therapeutic delivery vehicle.

The invention provides soluble ECM compositions and methods for treatment of conditions with endothelial cell injury or dysfunction, leaky vasculature, disrupted endothelial cell junctions, inhibited vasodilation, and inflammation.

The invention provides soluble ECM compositions and methods for treatment of conditions with potential reperfusion injury, including myocardial infarction, stroke, and peripheral artery and vascular disease. The soluble ECM compositions can serve as a tissue engineering scaffold to reduce reperfusion injury, reduce apoptosis, and promote tissue repair.

The invention provides soluble ECM compositions and methods for treatment of excessive or persistent reactive oxygen species (ROS) production/signaling resulting in endothelial cell activation and inflammation. The soluble ECM compositions can protect cells and tissues from ROS injury and inflammation through physical shielding and/or ROS sequestration.

The invention provides soluble ECM compositions and methods for treatment of heart disease, ischemia and perfusion. The soluble ECM compositions can promote neoangiogenesis and increase tissue perfusion.

The invention provides soluble ECM compositions and methods for treatment of diabetes, insulin resistance. The soluble ECM compositions can treat endothelial cells, restoring endothelium-dependent vasodilation.

The invention provides soluble ECM compositions and methods for treatment of cancer, including tumor growth, metastasis. The soluble ECM compositions can treat leaky vessels and endothelium dysfunction present in cancer. ECM degradation products have been shown to inhibit tumor growth and formation.

The invention provides soluble ECM compositions and methods for treatment of pulmonary diseases, such as chronic obstructive pulmonary disease, asthma, pulmonary artery hypertension. The soluble ECM compositions can be infused to treat injured tissues and/or endothelial cells of the lungs.

The invention provides soluble ECM compositions and methods for treatment of chronic kidney failure. The soluble ECM compositions can treat vessels to restore vasodilation and constriction.

The invention provides soluble ECM compositions and methods for treatment of venous thrombosis. The soluble ECM compositions infusion can coat vessels to prevent thrombosis and platelet aggregation.

The invention provides soluble ECM compositions and methods for treatment of severe infectious diseases, specifically diseases that have a disrupted endothelial barrier, such as hemorrhagic fever viruses including dengue hemorrhagic fever and hantavirus pulmonary syndrome. The soluble matrix infusions can treat and restore the endothelial barrier.

The invention provides soluble ECM compositions and methods for treatment of atherosclerosis. The soluble ECM compositions infusion can prevent plaque rupture by coating and stabilizing atherosclerotic plaques, or it can stick to endothelial cells and reduce inflammation.

The invention provides soluble ECM compositions and methods for treatment of liver cirrhosis, acute liver failure. The soluble ECM compositions can treat endothelial dysfunction in liver cirrhosis. The soluble ECM compositions can attenuate inflammation and oxidative stress.

The invention provides soluble ECM compositions and methods for treatment of tissue hemorrhage and edema. The soluble matrix can coat endothelial cells, fill gaps in the endothelial cell layer, increase tissue perfusion through vasostimulatory effects, or reduce fluid entering a tissue.

The invention provides soluble ECM compositions and methods for treatment of traumatic brain and other neurological injury. The soluble matrix can treat endothelial cells to repair leaky vessels, restore endothelial-dependent dilation and nitric oxide production, and reduce inflammation and oxidative stress.

Intracoronary infusion is an alternative approach to transendocardial injections. Intracoronary delivery can accompany a balloon angioplasty during the typical course of treatment for a myocardial infarction (MI). Intracoronary infusion utilizes the leaky vasculature following an acute MI, therefore permitting a biomaterial to enter the infarct region [5]. In the prior art formulation, the matrix material (MM) is composed of a soluble fraction and insoluble submicron particles (>800 nm), which are too large to pass through or stick to leaky vasculature. As a result, a method has been provided to at least partially isolate the soluble fraction, termed soluble MM (SolMM), which can pass through and/or coat the leaky vasculature and still form a hydrogel in vivo. Since the SolMM is derived from MM, the SolMM will have similar therapeutic effects, including decreased cardiomyocyte apoptosis, neovascularization, and reduced negative LV remodeling.

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to age-related eye diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

### EXAMPLES

### Experiment 1—SolMM Production and Characterization

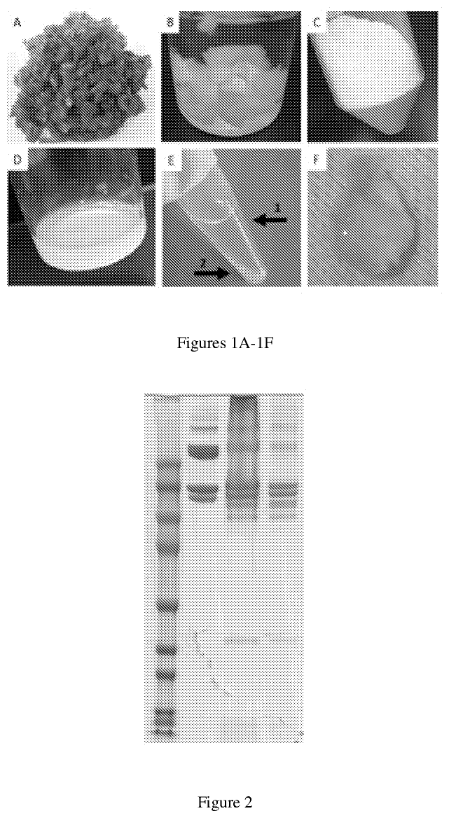

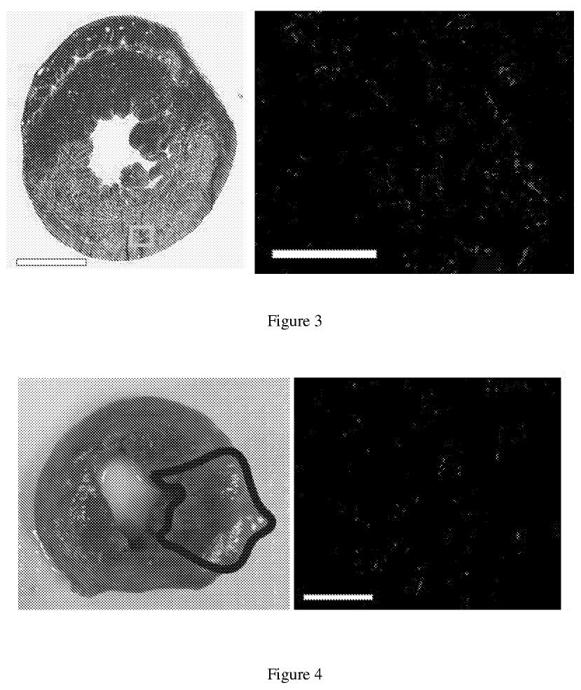

The formulation of myocardial matrix (MM) can be generated based on previously described protocols (FIG. 1) [3]. In brief, fresh hearts are harvested from pigs (approx. 30-45 kg) and the LV myocardium is isolated. Major vessels and connective tissue are removed, and the remaining tissue will be cut into pieces less than 5 mm³ (FIG. 1A). Tissue is decellularized in 1% (w/v) sodium dodecyl sulfate (SDS) for 4-5 days until the tissue is completely white, followed by an additional day of water rinsing to remove residual SDS (FIG. 1B). The material is lyophilized and milled into a fine powder (FIG. 1C) and subsequently partially enzymatically digested for 48 hours. The material is then neutralized and buffered to match in vivo conditions, yielding MM (FIG. 1D), capable of thermally induced gelation.

Next, the MM is centrifuged at 15,000 RCF at 4° C. to separate the soluble and insoluble fractions (FIG. 1E). The supernatant is isolated from the insoluble pellet, and this supernatant will be referred to as the soluble MM fraction (SolMM). SolMM is then dialyzed and lyophilized to adjust the concentration and ratio of salts to maintain physiological conditions for SolMM. The SolMM is then resuspended at a high concentration (16 mg/mL), passed through 0.22 μm filters into sterile containers, lyophilized, weighed, and stored at −80° C. until needed. The SolMM is then resuspended to the appropriate concentration in sterile water approximately 30 minutes before injection. This suspension can then gel upon subcutaneous injection in a rat within 5 minutes (FIG. 1F, 10 mg/mL, 500 μL). Material consistency can be assessed by polyacrylamide gel electrophoresis for protein distribution, Picogreen assay for DNA content, dimethylmethylene blue assay for sulfated glycosaminoglycan (sGAG) content, and methylene blue assay for SDS content. Due to the digestion process to generate the MM and the resulting SolMM, one cannot get accurate data from mass spectrometry. However, PAGE shows an overlapping distribution of proteins between MM and SolMM, excluding high molecular weight proteins in SolMM (FIG. 2, lane 4).

### Experiment 2—Hemocompatibility of the SolMM with Human Blood

The interaction between SolMM and human blood samples (n=4) is assessed at different dilutions (1:1, 1:2, 1:10) of SolMM to whole human blood or platelet rich plasma. 1:1 represents the highest possible ratio between blood and SolMM, whereas 1:10 represents a physiologically relevant dilution based on the volumetric flow rate of the coronary vasculature and intended infusion rate (1 ml/min). Hemocompatibility is assessed as previously described for MM [4]. Red blood cell aggregation will be performed within 4 hours of sampling on a Myrenne aggregonometer (Myrenne GmbH) after adjusting hematocrit to 45% with autologous plasma. Aggregation is assessed following stasis (M0) or a low shear rate (3 Hz; M1) while absorbance (800 nm) is measured for 5 seconds. Similarly, platelet aggregation is measured with isolated platelet rich plasma on a lumi-aggregonometer (Chrono-log). Using the same dilutions as above for sample to platelet rich plasma, high concentration coagulation cascade agonists (adenosine diphosphate, epinephrine, and collagen) is added (1:200-1:1000 dilutions), and platelet aggregation is measured via absorbance (600-620 nm).

Results from 1:1, and 1:10 dilutions (material to human blood) suggest that SolMM was hemocompatible, as all values fall within normal physiological ranges (Table 1).

TABLE 1

| | Standard | Saline | 1:1 Soluble | 1:10 Soluble |
|---|---|---|---|---|
| | Ranges | Control | matrix | matrix |
| Clotting time (s) | 20-36 | 24 ± 3 | 33 ± 3 | 26 ± 2 |
| Fibrinogen (mg/dl) | 100-200 | 163 ± 25 | 154 ± 20 | 161 ± 14 |
| Platelets (10³/mm³) | 100-200 | 145 ± 21 | 174 ± 13 | 146 ± 25 |

Platelet Aggregation

| | | | | |
|---|---|---|---|---|
| ADP 3 mM (%) | 60-80 | 64 ± 3 | 75 ± 4 | 67 ± 2 |
| ADP 1 mM (%) | 60-80 | 71 ± 2 | 77 ± 1 | 75 ± 3 |
| Epinephrine (%) | 60-80 | 67 ± 4 | 73 ± 2 | 75 ± 3 |
| Collagen (%) | 60-80 | 73 ± 5 | 76 ± 5 | 81 ± 4 |

Table 1 shows hemocompatibility of soluble matrix. 1:1 dilution of 10 mg/ml soluble matrix to human blood represents the highest ratio, whereas as the 1:10 dilution (1 mg/ml) represents a physiologically relevant dilution.

### Experiment 3—Distribution, Retention, and Efficacy in a Small Animal Ischemia-Reperfusion Model

Using a Sprague Dawley rat (225-250 g) ischemia-reperfusion model of MI, the left coronary artery is occluded for 45 minutes, followed by reperfusion. Within 5 minutes following reperfusion, the aorta is clamped for approximately 15 seconds to simulate intracoronary infusion, and 200 μl of SolMM is injected in to the LV lumen at a concentration of 6, 10, or 14 mg/mL. This will force the material into the coronary arteries and then distribute into the infarcted myocardium [12]. Hearts (n=2 per concentration) are isolated 60-minutes post-injection to determine if the material will initially distribute and then be retained in the heart, as non-gelling materials are cleared from the heart within an hour [5]. SolMM is conjugated with Alexa Fluor™ 568 N-hydroxysuccinimidyl ester (Invitrogen) to allow for fluorescent detection and analysis. Retention of the material is tested with the optimal concentration at the time points of 6, 12, and 24 hours, 2, 3, 4, and 5 days, and 1 week post-injection (n=2-3 per time point) to assess degradation. Saline (n=3 per time point) is mixed with unconjugated Alexa Fluor™ 568 to serve as a control. Hearts are fresh frozen in OCT Tissue-Tek compound and short-axis sectioned with 16 regions evenly spaced regions (approx 300 μm between regions), 4 slides per region in duplicate, and 10 μm per section. One slide per region is used for H&E to confirm infarction, and one slide per region is used for fluorescent analysis of the SolMM.

Distribution in the infarct regions increases with concentration, as indicated by a greater distribution of pre-labeled gels and increased intensity of the infarct region (10 mg/mL shown in FIG. 2) using concentrations of 6, 10, and 14 mg/mL 1 hour post-injection (n=2 per group); however, based on the yield of SolMM production, 10 mg/mL is used for future experiments. Yield is particularly limited based on the filtration step noted in Experiment 1, as SolMM was resuspended at 16 mg/mL and subsequently filtered resulting in approximate concentrations of 10 mg/mL. Resuspended concentrations above 16 mg/mL would typically not pass through the filter.

Based on the time course histology, material was observed in infarcted hearts up to approximately 3 days post-infusion.

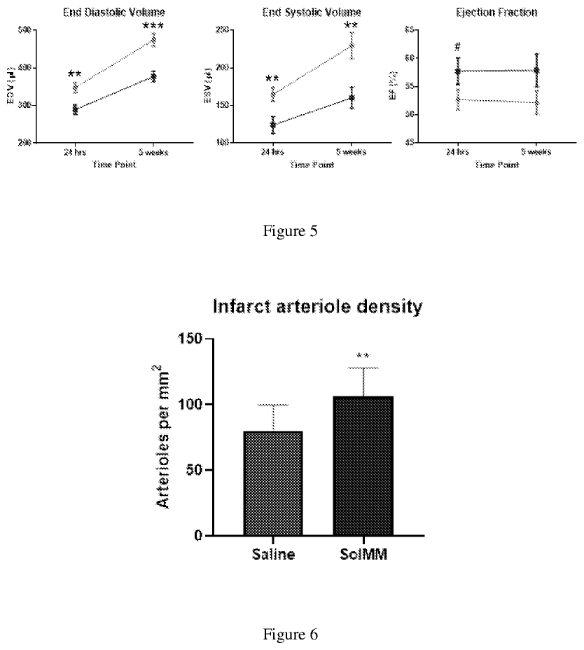

In a rat ischemia-reperfusion model, the left coronary artery was occluded for 35 minutes to simulate myocardial infarction. The heart was then reperfused, and soluble matrix was infused through the coronaries using an aortic-cross clamp model. Rats were imaged using magnetic resonance imaging 24 hours and 5 weeks following infusion. Left ventricular (LV) volumes and ejection fraction are shown in FIG. 5. Twenty-four hours followings infusion, significantly preserved LV volumes (end systolic and end diastolic) were observed over saline infused controls. Ejection fraction showed a trending increase over saline controls. Five weeks later, matrix infused LV volumes were also significantly reduced compared to saline infused controls, showing that a matrix infusion mitigates negative left ventricular remodeling.

### Experiment 4: Infusible Extracellular Matrix Using a Balloon Infusion Catheter to Repair the Heart Following Myocardial Infarction

Following a myocardial infarction, extracellular matrix was infused through a vessel (e.g. left anterior descending or left main artery) of the heart for targeted delivery using a balloon infusion catheter. FIG. 4 shows distribution and retention of soluble myocardial matrix (SolMM) 1 hour following intracoronary infusion in a porcine ischemia-reperfusion model using a balloon infusion catheter. FIG. 4 left shows a short axis gross histology of infarcted pig heart. Infarct outlined in blue greyscales. FIG. 4 right shows infarcted myocardium displaying SolMM micro-gels throughout the infarcted myocardium in the red greyscales channel.

Satellite organs (brain, kidney, liver, lung, spleen) were evaluated by a blinded histopathologist and did not show any abnormal signs of ischemia or inflammation 1 hour following matrix infusions (Table 2). Soluble matrix gels were not observed in any of the satellite organs, suggesting a targeting ability of infusible matrix to ischemic tissue.

TABLE 2

| | | Normal/Abnormal/ |
|---|---|---|
| PI Lab Animal ID/slide number | Tissue(s) | Autolysis |
| P050 | brain | N |
| | kidney | N |
| | liver | N |
| | lung | N |
| | spleen | N |
| P057 | brain | N |
| | kidney | N |
| | liver | N |
| | lung | N |
| | spleen | N |

Table 2 shows that intracoronary soluble matrix infusions in a porcine ischemia-reperfusion model shows no abnormal signs of ischemia or inflammation in satellite organs (brain, kidney, liver, lung, and spleen).

### Experiment 5: Infusible Matrix can be Used as a Scaffold to Promote Angiogenesis in Ischemic or Injured Tissues

FIG. 6 shows increased infarct arteriole density following matrix infusions in a myocardial infarction model. Infarcts were imaged 5 weeks following infusions in a rat ischemia-reperfusion model. Arterioles were identified by co-staining for alpha-smooth muscle actin and isolection and manually traced in ImageJ. Upregulation of angiogenic pathways are shown in FIG. 6.

### Experiment 6: Infusible Matrix can be Used as a Scaffold to Reduce Cell Apoptosis or Necrosis of Ischemic or Injured Tissues

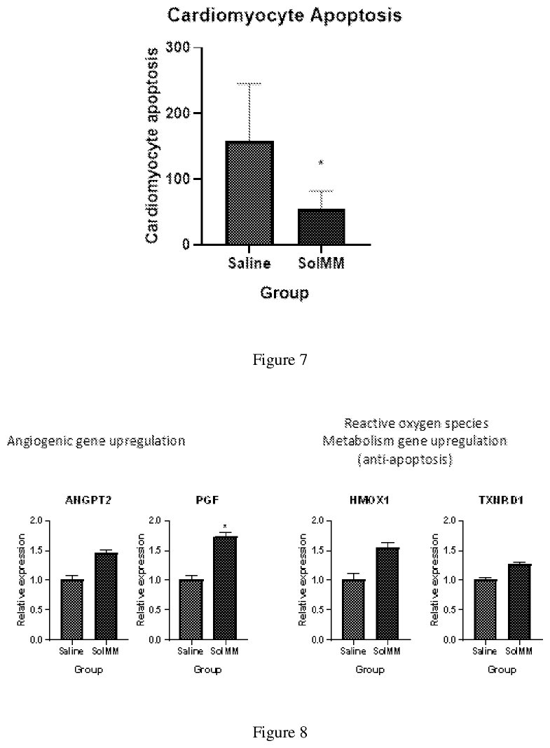

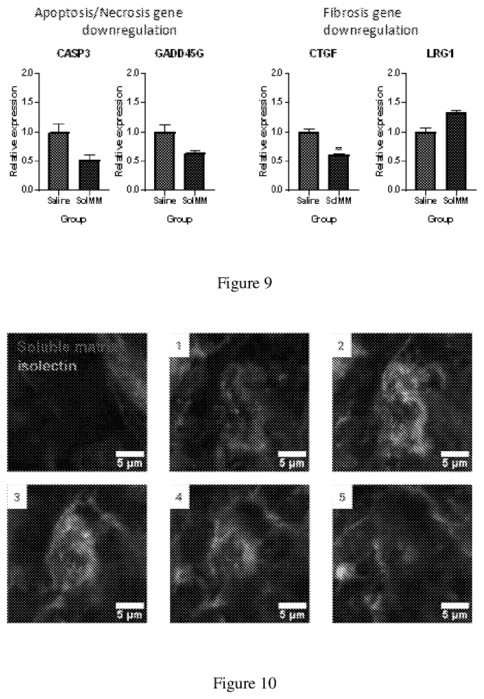

FIG. 7 shows decreased cardiomyocyte apoptosis following matrix infusions in a myocardial infarction model. Infarcts and infarct border zones were stained with alpha-actinin for cardiomyocytes and cleaved-caspase 3 for apoptosis. Apoptotic cardiomyocytes were manually counted in ImageJ. Decreased apoptosis could extend to other cells types, but not limited to endothelial cells, immune cells, fibroblasts, neurons, and (cardio)myocytes. Decreased apoptosis/necrosis pathways are shown in FIG. 9. Decreased apoptosis could be explained by increased reactive oxygen species (ROS) metabolism, as upregulated ROS metabolic pathways are shown in FIG. 8.

FIGS. 8 and 9 show differential gene expression, suggesting pathways for repair of infusible extracellular matrix therapies. RNA was isolated from left ventricular free wall tissue 1 day and 3 days following matrix infusion and ischemia-reperfusion injury. At day 1, angiogenic and reactive oxygen species metabolic pathways were upregulated. At day 3, decreased apoptosis/necrosis and decreased fibrotic pathways were observed. LRG1 downregulation is implicated in cardiac fibrosis, and a trend was observed in the opposite direction. Saline infusions were used as controls.

### Experiment 7: Matrix Infusions can Treat Endothelial Cell Injury/Dysfunction. Soluble Matrix can Coat Endothelial Cells to Reduce Reactive Oxygen Species Injury, Increase Endothelial Cell Survival, and/or Fill in Leaky Vasculature Gaps Following Ischemic Injury

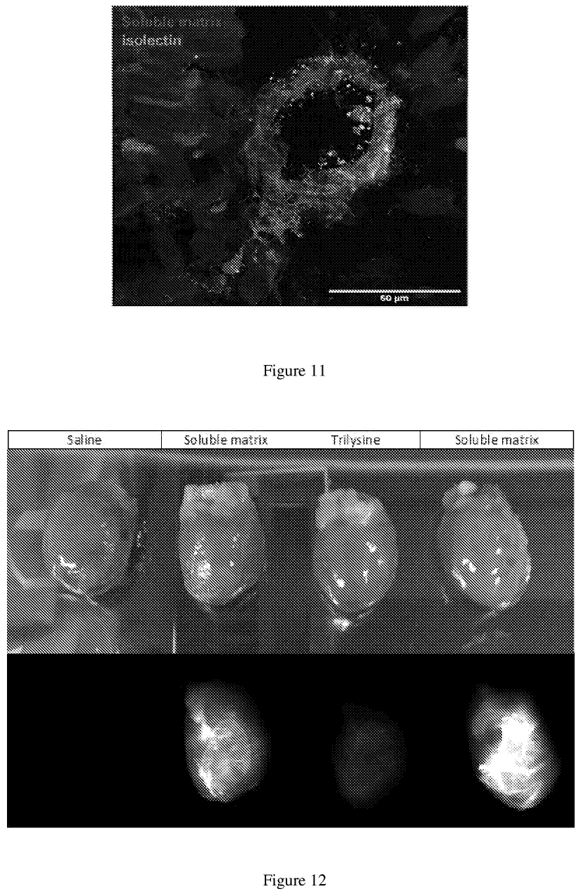

Following ischemic injury and matrix infusions, soluble matrix was observed to coat the lumen of small vessels (capillaries/endothelial cells). FIG. 10 shows an endothelial cell (green greyscales) lumen coated with soluble matrix (red greyscales). Note, that the matrix does not block the lumen. Additionally, FIG. 11 shows soluble matrix overlapping endothelial cells in a large vessel, while not blocking the lumen. Hearts were imaged using confocal microscopy up to 24 hours post-infusion and simulated myocardial infarction.

### Experiment 8: Infusible Matrix can be Co-Delivered with Drug, Growth Factor, microRNA, or Other Therapeutic Agent

The soluble extracellular matrix composition has potential binding domains for growth factors, microRNAs, and other potential drugs or therapeutics. As an infusible matrix can gel in tissue following an infusion, it can be used for slow release of a therapy.

FIG. 12 shows soluble ECM retention in infarcted tissue 24 hours following matrix infusion in an ischemia-reperfusion model. From left to right, hearts were infused with saline, matrix conjugated w/VivoTag 750, trilysine conjugated w/VivoTag750, and matrix conjugated w/VivoTag 750. Twenty four hours following infusion, hearts were harvested and imaged on a Licor Odyssey. Matrix infused hearts showed greater signal intensity as opposed to saline infused and trilysine infused hearts. Trilysine with VivoTag 750 was used as a small peptide control and showed no appreciable retention.

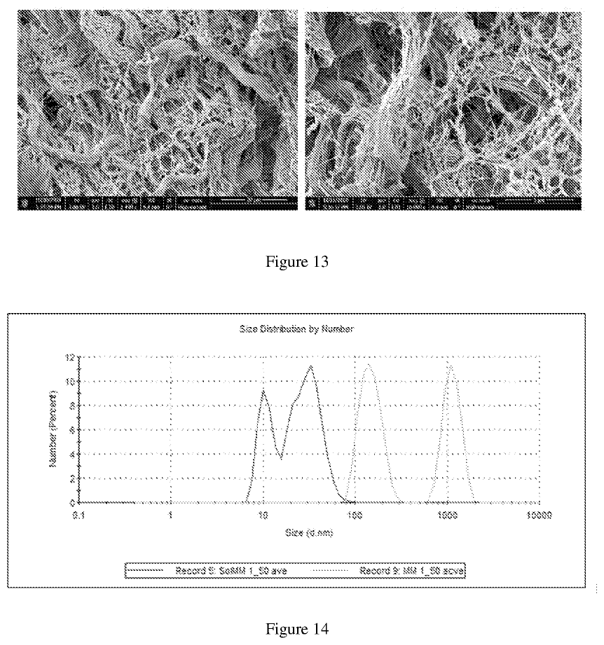

FIG. 13 shows the nanofibrous architecture of a soluble matrix hydrogel. A 10 mg/ml pre-gel solution was subcutaneously injected into the back of a rat, which then formed a gel and was harvested for scanning electron microscopy imaging. Gel structure is reminiscent of native extracellular matrix.

### Experiment 9: Dynamic Light Scattering Analysis Shows Difference Between MM and SolMM

FIG. 14 shows dynamic light scattering data for soluble matrix (SolMM) and full matrix (MM) at 1:50 dilutions (1.0 mg/ml & 0.6 mg/ml respectively), showing soluble matrix particles less than 100 nm in diameter whereas full matrix has larger particles.

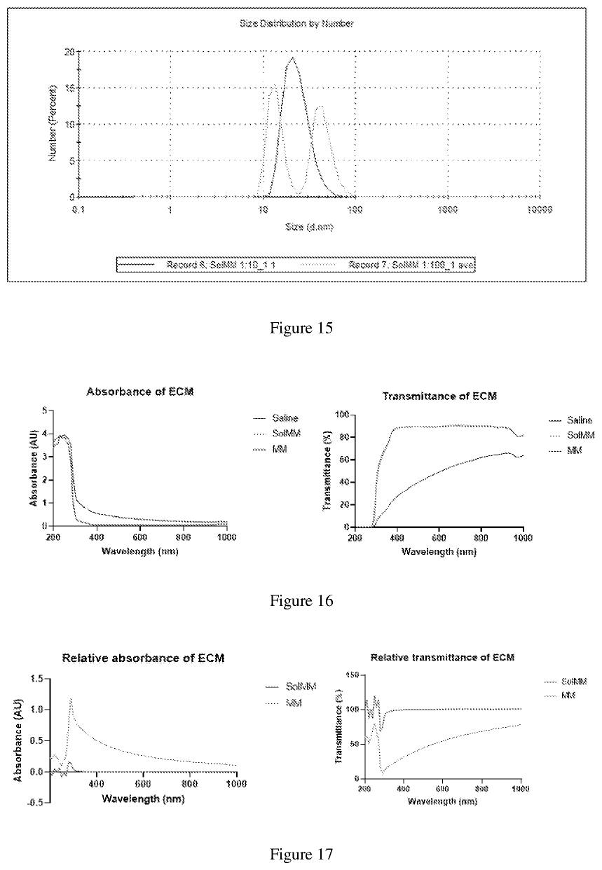

FIG. 15 shows dynamic light scattering data for soluble matrix (SolMM) at 1:10 and 1:100 dilutions (1.0 mg/ml & 0.1 mg/ml respectively), showing particles less than 100 nm in diameter.

FIG. 16 shows absorbance (left) and transmittance (right) of saline, soluble matrix (SolMM), and full matrix (MM).

FIG. 17 shows relative absorbance (left) and transmittance (right) of soluble matrix (SolMM) and full matrix (MM).

### REFERENCES

[1] Benjamin et al., American Heart Association Statistics, O.b.o.t.A.H.A.S.C. Stroke Statistics Subcommittee, S. Stroke, Heart Disease and Stroke Statistics-2017 Update: A Report From the American Heart Association, Circulation 135(10) (2017) e146-e603.
[2] Singelyn, et al., Naturally derived myocardial matrix as an injectable scaffold for cardiac tissue engineering, Biomaterials 30(29) (2009) 5409-16.
[3] Singelyn, et al., Catheter-deliverable hydrogel derived from decellularized ventricular extracellular matrix increases endogenous cardiomyocytes and preserves cardiac function post-myocardial infarction, Journal of the American College of Cardiology 59(8) (2012) 751-63.
[4] Seif-Naraghi, et al., Safety and efficacy of an injectable extracellular matrix hydrogel for treating myocardial infarction, Science translational medicine 5(173) (2013) 173ra25.
[5] Nguyen, et al., Enzyme-Responsive Nanoparticles for Targeted Accumulation and Prolonged Retention in Heart Tissue after Myocardial Infarction, Advanced Materials 27(37) (2015) 5547-5552.
[6] Wassenaar, et al., Evidence for mechanisms underlying the functional benefits of a myocardial matrix hydrogel for post-MI treatment, Journal of the American College of Cardiology 67(9) (2016) 1074-86.
[7] Schuster, et al., Expansion of transmural myocardial infarction: a pathophysiologic factor in cardiac rupture, Circulation 60(7) (1979) 1532-1538.
[8] Arsenos, et al., Arrhythmic sudden cardiac death: substrate, mechanisms and current risk stratification strategies for the post-myocardial infarction patient, Hellenic J Cardiol 54(4) (2013) 301-315.
[9] Leor, et al., Intracoronary Injection of In Situ Forming Alginate Hydrogel Reverses Left Ventricular Remodeling After Myocardial Infarction in Swine, Journal of the American College of Cardiology 54(11) (2009) 1014-1023.
[10] Frey, et al., Intracoronary delivery of injectable bioabsorbable scaffold (IK-5001) to treat left ventricular remodeling after ST-elevation myocardial infarction: A first-in-man study, Circulation: Cardiovascular Interventions 7(6) (2014) 806-812.
[11] Spang, et al., Extracellular matrix hydrogel therapies: In vivo applications and development, Acta Biomater. 68 (2018) 1-14.
[12] Cheng, et al., Magnetic enhancement of cell retention, engraftment, and functional benefit after intracoronary delivery of cardiac-derived stem cells in a rat model of ischemia/reperfusion, Cell transplantation 21(6) (2012) 1121-35.
[13] Rane, et al., Increased infarct wall thickness by a bio-inert material is insufficient to prevent negative left ventricular remodeling after myocardial infarction, PloS one 6(6) (2011) e21571.
[14] Conesa, et al., A survey of best practices for RNA-seq data analysis, Genome biology 17 (2016) 13-13.
[15] Costa-Silva, et al., RNA-Seq differential expression analysis: An extended review and a software tool, PLOS ONE 12(12) (2017) e0190152-e0190152.
[16] Wang, et al., Humanized mouse model for assessing the human immune response to xenogeneic and allogeneic decellularized biomaterials, Biomaterials 129 (2017) 98-110.
[17] Sonnenberg, et al., Delivery of an engineered HGF fragment in an extracellular matrix-derived hydrogel prevents negative LV remodeling post-myocardial infarction, Biomaterials 45 (2015) 56-63.
[18] Seif-Naraghi, et al., Injectable extracellular matrix derived hydrogel provides a platform for enhanced retention and delivery of a heparin-binding growth factor, Acta Biomater. 8(10) (2012) 3695-703.
[19] van den Akker, et al., Intramyocardial stem cell injection: go(ne) with the flow, European heart journal (2016) ehw056-ehw056.
[20] Wassenaar, et al., Modulating in vivo degradation rate of injectable extracellular matrix hydrogels, J. Mater. Chem. B 4(16) (2016) 2794-2802.
[21] Grover, et al., Myocardial matrix-polyethylene glycol hybrid hydrogels for tissue engineering, Nanotech. 25(1) (2014) 014011.
[22] Gallet, et al., Exosomes secreted by cardiosphere-derived cells reduce scarring, attenuate adverse remodelling, and improve function in acute and chronic porcine myocardial infarction, European Heart Journal 38(3) (2016) ehw240-ehw240.
[23] Dawn, et al., Cardiac stem cells delivered intravascularly traverse the vessel barrier, regenerate infarcted myocardium, and improve cardiac function, Proceedings of the National Academy of Sciences 102(10) (2005) 3766-3771.
[24] Gallet, et al., Intracoronary delivery of self-assembling heart-derived microtissues (cardiospheres) for prevention of adverse remodeling in a pig model of convalescent myocardial infarction, Circ Cardiovasc Interv. 8(5). (2015) e002391. doi: 10.1161/CIRCINTERVENTIONS.115.002391-e002391. doi: 10.1161/CIRCINTERVENTIONS.115.002391.
[25] Bolli, et al., Intracoronary Delivery of Autologous Cardiac Stem Cells Improves Cardiac Function in a Porcine Model of Chronic Ischemic Cardiomyopathy, Circulation 128(2) (2013) 122-131.
[26] McKay, Raymond G., et al, “Left ventricular remodeling after myocardial infarction: a corollary to infarct expansion.” Circulation 74, no. 4 (1986): 693-702.
[27] Harpster, Mark H., et al. “Earliest changes in the left ventricular transcriptome post-myocardial infarction.” Mammalian genome 17, no. 7 (2006): 701-715.

What is claimed is:

1. A method of preparing a soluble extracellular matrix (ECM) composition, comprising:
a. digesting decellularized ECM material with an acid protease;
b. neutralizing the digested ECM material in liquid to a pH of 7.0-8.0;
c. processing the liquid ECM to produce soluble and insoluble fractions; and
d. separating at least a portion of the soluble fraction from the insoluble fraction, to yield a soluble ECM composition.

2. The method of claim 1, wherein the processing the liquid ECM to produce the soluble and insoluble fractions is performed by centrifugation.

3. The method of claim 1, wherein the processing the liquid ECM to produce the soluble and insoluble fractions is performed by dialysis or filtration.

4. The method of claim 1, wherein the separating is performed with a 250 nm or smaller size exclusion filter.

5. The method of claim 1, wherein the soluble ECM composition is further lyophilized and re-hydrated.

6. A soluble ECM composition comprising decellularized, digested and neutralized tissue having at least a portion of solid ECM materials removed therefrom, wherein the soluble ECM composition passes through a 250 nm size exclusion filter.

7. The soluble ECM composition of claim 6, wherein the composition is formulated for intravascular infusion.

8. The soluble ECM composition of claim 6, wherein the composition is a liquid at room temperature and form a gel following infusion or injection in vivo.

9. The soluble ECM composition of claim 6, wherein the composition is a liquid at room temperature and forms a coating lining damaged blood vessels following infusion or injection in vivo.

10. The soluble ECM composition of claim 6, wherein the composition is a liquid at room temperature and fills the pores between endothelial cells following infusion or injection in vivo.

11. The soluble ECM composition of claim 6, derived from human, animal, embryonic, or fetal tissues.

12. The soluble ECM composition of claim 6, derived from heart, brain, bladder, small intestine, or skeletal muscle tissues, kidney, liver, lung, and blood vessel.

13. A method of treating a subject to promote tissue repair, comprising administering to a subject in need thereof an effective amount of an infusion of the soluble ECM composition of claim 6.

14. The method of claim 13, wherein the infusion is delivered through a catheter, intravenously, or intravascularly.

15. The method of claim 13, wherein when delivered in vivo, the soluble ECM composition forms a gel in tissue.

16. The method of claim 13, wherein the soluble ECM composition is crosslinked with glutaraldehye, formaldehyde, bis-NHS molecules, or other crosslinkers before administration.

17. The method of claim 13, wherein the soluble ECM composition is combined with cells, peptides, proteins, DNA, drugs, nanoparticles, nutrients, survival promoting additives, proteoglycans, and/or glycosaminoglycans before administration.

18. The method of claim 13, wherein the soluble ECM composition is combined and/or crosslinked with a synthetic polymer or biologically derived material before administration.

19. The method of claim 13, wherein the soluble ECM composition causes endogenous cell ingrowth, angiogenesis, and regeneration in the subject.

20. The method of claim 13, wherein the soluble ECM composition promotes cell survival and reduces inflammation in the subject.

21. A method for treating acute myocardial infarction comprising injecting or infusing in a subject in need an effective amount of a soluble ECM composition comprising decellularized, digested and neutralized tissue having at least a portion of solid ECM materials removed therefrom.

22. The method of claim 21, wherein said composition is delivered intravascularly.

23. The method of claim 21, wherein said composition is delivered with a balloon infusion catheter.

24. The method of claim 21, wherein said composition transitions to a gel form in tissue after delivery.

25. The method of claim 21, wherein the composition is a liquid at room temperature and forms a coating lining infarct blood vessels after delivery.

26. The method of claim 21, wherein the composition is a liquid at room temperature and fills the pores between infarct endothelial cells after delivery.

27. The method of claim 21, wherein said composition degrades within one to 14 days following injection or infusion.

28. The method of claim 21, wherein injection or infusion of said composition repairs damage to cardiac muscle sustained by said subject.

29. The method of claim 21, wherein injection or infusion of said composition repairs damage caused by ischemia in said subject.

30. The method of claim 21, wherein said effective amount is an amount that increases blood flow, increases viable tissue mass, or induces new vascular formation in the area of the injection or infusion of the subject.

31. A method of treating endothelial cell injury and/or dysfunction comprising injecting or infusing in a subject in need an effective amount of a soluble ECM composition comprising decellularized, digested and neutralized tissue having at least a portion of solid ECM materials removed therefrom.

32. The method of claim 31, wherein said effective amount promotes endothelial cell survival, proliferation, or vasoactivity and/or decreases inflammation, apoptosis, reactive oxygen species injury, or leaky vasculature.
